(19) United States
(12) Patent Application Publication
(10) Pub. No.: US 2026/0026725 A1
(43) Pub. Date: Jan. 29, 2026

(54) LIVING BODY TACKY AGENT AND BIOSENSOR

(71) Applicant: NITTO DENKO CORPORATION, Ibaraki-shi, Osaka (JP)

(72) Inventors: Chiharu YANO, Ibaraki-shi, Osaka (JP); Kaori AKAMATSU, Ibaraki-shi, Osaka (JP)

(21) Appl. No.: 18/997,727

(22) PCT Filed: Jul. 21, 2023
(86) PCT No.: PCT/JP2023/026884

§ 371 Date: Jan. 22, 2025

(30) Foreign Application Priority Data
Jul. 26, 2022 (JP) 2022-118682
Mar. 16, 2023 (JP) 2023-042213

(51) Int. Cl. A61B 5/257; A61L 24/04

(52) U.S. Cl. CPC A61B 5/257; A61L 24/046

(57) ABSTRACT

A living body tacky agent according to the present invention is a living body tacky agent pasted on a living body. A peel tackiness of the living body tacky agent when the living body tacky agent, which is pasted on an adherend having an Asker C hardness of 0, is peeled from the adherend at an angle of 60° between a surface of the living body tacky agent facing the adherend and a pasting surface of the adherend is 5.0 N/cm or greater.

### TECHNICAL FIELD

The present invention relates to a living body tacky agent, and a biosensor.

### BACKGROUND ART

Wearable biosensors for acquiring biological information such as electrocardiogram waveforms, pulse waves, brain waves, myoelectric potentials, and the like are used in medical institutions such as hospitals, clinics, and the like, nursing facilities, households, and the like. A biosensor is equipped with: a living body tacky agent having tackiness and provided on a surface of a substrate to be faced with a living body; and a biological electrode to be brought into contact with a living body for acquiring biological information of a subject. When measuring biological information, the living body tacky agent is pasted on the skin of the subject such that the biological electrode acquires an electric signal related to the biological information. As such a living body tacky agent, for example, a pressure-sensitive tacky layer made of a mixture containing isononyl acrylate (INA), 2-methoxyethyl acrylate (2-MEA), and acrylic acid is disclosed. The pressure-sensitive tacky layer is pasted on the pasting side of the substrate layer of the biosensor, and is directly pasted on the skin when using the biosensor (see, for example, PTL 1).

### CITATION LIST

### PATENT LITERATURE

PTL 1: Japanese Patent Application Laid-Open Publication No. 2020-163120

### SUMMARY OF THE INVENTION

### Technical Problem

Here, in general, the tackiness of such a living body tacky agent as the pressure-sensitive tacky layer of PTL 1 is evaluated according to Japanese Industrial Standards (JIS) Z 0237:2009. For example, the tackiness of the living body tacky agent on a test plate is evaluated by a test method of pasting the living body tacky agent on a hard test plate such as a bakelite plate, a stainless steel plate, or the like, and peeling the living body tacky agent at 180° with respect to the test plate, to select a material that is optimum as the living body tacky agent.

However, the test plate is smooth and rigid, and its surface shape does not deform, whereas a living body surface has multiple minute undulations and the living body surface easily deforms in response to a body movement. Therefore, since the tackiness of the living body tacky agent tends to deviate between the case in which the living body tacky agent is pasted on the test plate and the case in which it is pasted on the surface of a living body, the living body tacky agent may not exhibit a sufficient tackiness on the surface of the living body even if it exhibits excellent tackiness on the test plate. Biosensors are often used by being pasted on the surface of a living body such as the skin of a subject for a long time (for example, 24 hours or longer). Therefore, in order to acquire electric signals related to biological information of the subject for a long time stably, it is important that the living body tacky agent can reliably exhibit tackiness on the surface of the living body. An object of an embodiment of the present invention is to provide a living body tacky agent capable of exhibiting excellent adhesion reliability on the surface of a living body.

### Solution to the Problem

An embodiment of the present invention is a living body tacky agent to be pasted on a living body. A peel tackiness of the living body tacky agent when the living body tacky agent, which is pasted on an adherend having an Asker C hardness of 0, is peeled from the adherend at an angle of 60° between a surface of the living body tacky agent facing the adherend and a pasting surface of the adherend is 5.0 N/cm or greater.

### Advantageous Effects of the Invention

One embodiment of the present invention can exhibit excellent adhesion reliability on a living body surface.

### BRIEF DESCRIPTION OF THE DRAWINGS

FIG. 1 is an oblique view showing the overall configuration of a biosensor to which a living body tacky agent according to an embodiment of the present invention is applied.

FIG. 2 is a plan view showing an example of the components of the biosensor.

FIG. 3 is a cross-sectional view of the biosensor in the longer direction and is a cross-sectional view taken along I-I of FIG. 1.

FIG. 4 is an illustrative view showing the biosensor of FIG. 1 pasted on the chest of a living body.

FIG. 5 is a drawing showing the measurement results of 60° peel tackiness when a human skin gel sheet stored for three days under normal temperature and normal humidity conditions (22° C., 50 RH %) and high temperature and high humidity conditions (60° C., 90 RH %) was applied to an adherend.

FIG. 6 is a drawing showing the relationship between the number of times taken until the biosensors of Examples and Comparative Example are peeled, and the 60° peel tackiness of the living body tacky agent laminates used in Examples and Comparative Example.

### DETAILED DESCRIPTION OF THE INVENTION

Embodiments of the present invention will be described in detail below. In order to facilitate understanding of the description, the same reference numerals will be assigned to the same components in the drawings, and duplicate explanations will be omitted. Also, the components in the drawings may not be to scale. In this specification, the term “to” indicating a numerical range means that the numerical values described before and after the term are included as the lower limit and upper limit, unless otherwise particularly noted.

### Living Body Tacky Agent

The living body tacky agent according to this embodiment will be described below. The term “living body” refers to human bodies (humans) and animals such as cows, horses, pigs, roosters or hens, dogs, cats, and the like. The living body tacky agent according to this embodiment can be suitably used for living bodies, especially for human bodies. In this embodiment, a case where the living body is a human will be described as an example.

The living body tacky agent according to this embodiment is a tackifier used to be pasted on a part of a living body (for example, skin, scalp, forehead, and the like), and has a sheet-like shape. The living body tacky agent according to this embodiment may be formed in any shape such as substantially a rectangular shape, substantially a polygonal shape, substantially a circular shape, or substantially an elliptical shape in a plan view.

The living body tacky agent according to this embodiment has a peel tackiness of 5.0 N/cm or greater when the living body tacky agent according to this embodiment, when pasted on an adherend having an Asker C hardness of 0, is peeled from the adherend at a peeling angle of 60° with respect to the adherend. The peel tackiness is preferably 6.5 N/cm or greater and more preferably 7.5 N/cm or greater.

In this embodiment, the peeling angle refers to the angle between a surface of the living body tacky agent that faces the adherend and a pasting surface of the adherend, and refers to the angle between the surface of the living body tacky agent facing the adherend and the pasting surface of the adherend in an initial period before the living body tacky agent ends up being peeled from the adherend.

In this embodiment, the peel tackiness is also referred to as peel force (peel strength). The peel tackiness when peeling from the adherend at a peeling angle of 60° is also referred to as 60° peel tackiness. When measuring the peel tackiness, the temperature and humidity may be normal temperature and normal humidity (for example, 22° C., 50 RH %).

When measuring the peel tackiness, the peeling speed at which the living body tacky agent is peeled from the adherend may be appropriately set according to the size, type, and the like of the living body tacky agent used, and may be, for example, 150 mm/min.

A tendency the inventor has noticed in use of a living body tacky agent by pasting it on the surface of a living body is a mismatch with the peel tackiness in actual use of the living body tacky agent by pasting it on the skin of a subject, depending on the peeling angle of the living body tacky agent. The inventor has found that a living body tacky agent that has a peel tackiness at a peeling angle of 60° (60° peel tackiness) of 5.0 N/cm or greater with respect to an adherend having an Asker C hardness of 0 would be able to reliably exhibit the required adhesive force to the skin of a subject, with reduced deviation from the peel tackiness in actual pasting of the living body tacky agent on the surface of the subject's skin.

The angle by which the skin of a subject is deformed due to a body movement is often approximately 60° or less, and the peeling angle of 60° can include the maximum angle by which the skin would be deformed due to the subject's typical behaviors in life. Therefore, the peel tackiness of the living body tacky agent according to this embodiment with respect to the adherend at the peeling angle of 60° tends to be a value close to the peel tackiness which the living body tacky agent according to this embodiment possesses when it is actually pasted on the skin of the subject. Therefore, as long as having a peel tackiness at a peeling angle of 60° of 5.0 N/cm or greater, which matches the peel tackiness required for actual peeling from the surface of the subject's skin, the living body tacky agent according to this embodiment can possess a peel tackiness close to that for actual use, and can therefore exhibit excellent adhesion reliability to the skin surface.

The peel tackiness of the living body tacky agent according to this embodiment with respect to the adherend having an Asker C hardness of 0 at a peeling angle of 60° is determined by measuring the peel tackiness when, after the living body tacky agent according to this embodiment is pasted on a human skin gel sheet, peeling the living body tacky agent according to this embodiment, starting from one longer-direction end of the living body tacky agent according to this embodiment, from the human skin gel sheet at a peeling angle of 60° between a surface of each living body tacky agent on the human skin gel sheet side and the human skin gel sheet. Any human skin gel sheet that has an Asker C hardness of 0 may be used. A commercially available product can be used as the human skin gel sheet, and, for example, H0-1 available from Exseal Corporation may be used.

For the peel tackiness of the living body tacky agent according to this embodiment, a laminate (living body tacky agent) in which a plate-like support is pasted and laminated on a surface of the living body tacky agent according to this embodiment different from a surface thereof on the adherend side may be used, and a measured value obtained when the living body tacky agent according to this embodiment pasted on the adherend is peeled from the adherend, starting from one longer-direction end of the laminate, may be adopted. The support may be, for example, polyethylene terephthalate (PET) or the like. When PET or the like is used as the support, the peel tackiness of the living body tacky agent can be more accurately determined.

As described later, the 60° peel tackiness of the living body tacky agent according to this embodiment can be adjusted mainly based on the glass transition temperature Tg of the living body tacky agent according to this embodiment, and can also be adjusted based on the polymer molecular weight, gel fraction, saturated moisture content, components contained in the living body tacky agent and their contents, and the like.

The adhesion reliability to the skin surface can be evaluated by performing a tensile endurance test or the like.

When performing a tensile endurance test, for example, a high-performance artificial skin model (Product name: Bioskin plate, available from Beaulax Corporation) is used as the substitute for the skin, and a biosensor formed using the living body tacky agent according to this embodiment is pasted and fixed on the Bioskin plate. The Bioskin plate on which the biosensor is pasted is set in a typical surface-state tensile tester such as a small-size tabletop endurance tester, and the Bioskin plate is set such that the strain thereof becomes a predetermined value (for example, 20%), and is repeatedly stretched every 3 seconds until peeling occurs at an end of the biosensor. The tensile endurance can be evaluated by investigating the number of times (the number of repetitions) of stretching taken until peeling occurs.

The glass transition temperature Tg of the living body tacky agent according to this embodiment is preferably −57° C. or higher, more preferably −57° C. or higher, and more preferably −45° C. or higher. When the glass transition temperature Tg of the living body tacky agent according to this embodiment is −55° C. or higher, the living body tacky agent can easily click with an adherend having an Asker C hardness of 0, such as a human skin gel sheet, and can likewise click with and become pasted on the skin of a subject. The upper limit of the glass transition temperature Tg of the living body tacky agent according to this embodiment is not particularly limited. As long as it is −30° C. or lower, the living body tacky agent can maintain the state of clicking with an adherend having an Asker C hardness of 0, and can therefore likewise click with the skin of a subject and maintain a pasted state.

The polymer molecular weight of the living body tacky agent according to this embodiment can be appropriately selected according to the type of the material forming the living body tacky agent and the like, and may be, for example, from 500,000 to 1.8 million.

The gel fraction of the living body tacky agent according to this embodiment is not particularly limited, but may be 25% by mass to 65% by mass. When the gel fraction is 25% by mass to 65% by mass, increase in the peel tackiness of the living body tacky agent according to this embodiment over time is avoided, and contamination of the surface of the living body when peeling the living body tacky agent according to this embodiment from the surface of the living body is avoided. This facilitates peeling. Therefore, the living body tacky agent according to this embodiment has excellent peelability and handleability.

The saturated moisture content in the living body tacky agent according to this embodiment can be appropriately selected according to the type of the material forming the living body tacky agent, and the like, and may be, for example, 0.25% to 0.55% at 22° C. and 50% RH, and may be 0.50% to 1.3% at 40° C. and 92% RH.

The living body tacky agent according to this embodiment may have moisture permeability. Thus, water vapor due to sweat or the like generated from the skin on which the living body tacky agent according to this embodiment is pasted can be released to the outside through the living body tacky agent according to this embodiment.

The moisture permeability of the living body tacky agent according to this embodiment may be, for example, 1 g/(m²·day) or greater. The moisture permeability of the living body tacky agent according to this embodiment may be 10,000 g/(m²·day) or less.

When the moisture permeability of the living body tacky agent is 1 g/(m²·day) or greater, sweat or the like propagating through the living body tacky agent when the living body tacky agent is pasted on the skin can permeate the living body tacky agent to the outside, thereby reducing the load on the skin.

As the material for forming the living body tacky agent according to this embodiment, a material having pressure sensitive adhesiveness may be used. As the material having pressure sensitive adhesiveness, for example, an acrylic-based adhesive, a silicone-based adhesive, or the like may be used, and it is preferable to use an acrylic-based adhesive. An example of the acrylic-based adhesive is an acrylic polymer or the like described in Japanese Patent Application Laid-Open Publication No. 2002-65841.

The living body tacky agent according to this embodiment contains a polymer as a main component and may include additives such as a tackifier, a liquid component, a crosslinking agent, and the like as appropriate.

The polymer used in the living body tacky agent according to this embodiment is not particularly limited, and polymers having pressure-sensitive adhesiveness generally used for adhesives such as acrylic polymers and silicone-based polymers may be used, and it is preferable to use acrylic polymers.

The acrylic-based pressure-sensitive adhesive contains an acrylic polymer as a main component. A polymer obtained by polymerizing a monomer component containing (meth) acrylate ester such as isononyl acrylate, methoxyethyl acrylate, or the like as a main component may be used. The content of the main component in the monomer component is 60% by mass to 99% by mass, and the content of any optional component in the monomer component is 1% by mass to 40% by mass. As the acrylic polymer, for example, a (meth) acrylate ester-based resin described in Japanese Patent Application Laid-Open Publication No. 2003-42541 can be used.

The content of the polymer can be suitably selected, and may be, for example, 90 parts by mass to 120 parts by mass relative to 100 parts by mass of the living body tacky agent.

The tackifier is not particularly limited, and a common tackifier can be used. For example, terpene resin and the like may be used. Commercially available products may be used as tackifier resins. Specifically, Haritack PCJ available from Harima Chemicals, Inc., KE-311 available from Arakawa Chemical Industries, Ltd., and the like may be used.

The content of the tackifier can be appropriately selected, and may be, for example, 3 parts by mass to 30 parts by mass relative to 100 parts by mass of the living body tacky agent.

The liquid component is not particularly limited, and common liquid components can be used. For example, caprylic triglyceride and the like may be used. Commercially available products can also be used as the liquid component. Specifically, Coconade RK available from Kao Corporation and the like may be used.

The content of the liquid component can be appropriately selected, and may be, for example, 10 parts by mass to 20 parts by mass relative to 100 parts by mass of the living body tacky agent.

The crosslinking agent is not particularly limited, and common crosslinking agents such as isocyanate-based crosslinking agents and the like can be used. For example, polyfunctional isocyanate compounds and the like may be used.

The polyfunctional isocyanate compound is not particularly limited as long as it is, for example, a compound having preferably at least two or more isocyanate groups, and more preferably three or more isocyanate groups in the molecule. Specific examples include aliphatic polyisocyanates, alicyclic polyisocyanates, aromatic polyisocyanates and the like.

One of these may be used, or two or more of these can be used in combination.

Examples of the aliphatic polyisocyanates include 1,2-ethylenediisocyanate, tetramethylenediisocyanate such as 1,2-tetramethylenediisocyanate, 1,3-tetramethylenediisocyanate, 1,4-tetramethylenediisocyanate, and the like, hexamethylenediisocyanate such as 1,2-hexamethylenediisocyanate, 1,3-hexamethylenediisocyanate, 1,4-hexamethylenediisocyanate, 1,5-hexamethylenediisocyanate, 1,6-hexamethylenediisocyanate, 2,5-hexamethylenediisocyanate, and the like, 2-methyl-1,5-pentanediisocyanate, 3-methyl-1, 5-pentanediisocyanate, lysine diisocyanate, and the like.

Examples of the alicyclic polyisocyanates include isophorone diisocyanate, cyclohexyl diisocyanate such as 1,2-cyclohexyl diisocyanate, 1,3-cyclohexyl diisocyanate, 1,4-cyclohexyl diisocyanate, and the like, cyclopentyl diisocyanate such as 1,2-cyclopentyl diisocyanate, 1,3-cyclopentyl diisocyanate, and the like, hydrogenated xylylene diisocyanate, hydrogenated tolylene diisocyanate, hydrogenated diphenylmethane diisocyanate, hydrogenated tetramethyl xylene diisocyanate, 4,4′-dicyclohexyl methane diisocyanate, and the like.

Examples of the aromatic polyisocyanates include 2,4-tolylenediisocyanate, 2,6-tolylenediisocyanate, 4,4′-diphenylmethane diisocyanate, 2,4′-diphenylmethane diisocyanate, 2,2′-diphenylmethane diisocyanate, 4,4′-diphenyl ether diisocyanate, 2-nitrodiphenyl-4,4′-diisocyanate, 2,2′-diphenylpropane-4,4′-diisocyanate, 3,3′-dimethyldiphenylmethane-4,4′-diisocyanate, 4,4′-diphenylpropane diisocyanate, m-phenylenediisocyanate, p-phenylenediisocyanate, naphthylene-1,4-diisocyanate, naphthylene-1,5-diisocyanate, 3,3′-dimethoxydiphenyl-4,4′-diisocyanate, xylylene-1,4 diisocyanate, xylylene-1,3 diisocyanate, and the like.

As the polyfunctional isocyanate compound, dimers and trimers of aliphatic polyisocyanates, alicyclic polyisocyanates, aromatic polyisocyanates, and aromatic aliphatic polyisocyanates can be used. Specific examples include diphenylmethane diisocyanate dimers and trimers, reaction products of trimethylolpropane and tolylene diisocyanate, reaction products of trimethylolpropane and hexamethylene diisocyanate, polymers such as polymethylene polyphenyl isocyanate, polyether polyisocyanate, polyester polyisocyanate, and the like.

Examples of the reaction products of trimethylolpropane and tolylene diisocyanate include an adduct of trimethylolpropane with a tolylene diisocyanate trimer. Examples of the reaction products include an adduct of trimethylolpropane with hexamethylene diisocyanate trimer.

Commercially available products can be used as polyfunctional isocyanate compounds. Specific examples include the product name “Coronate L” (available from Nippon Polyurethane Industry Co., Ltd.) as the adduct of trimethylolpropane with tolylene diisocyanate trimer, the product name “Coronate HL” (available from Nippon Polyurethane Industry Co., Ltd.) as the adduct of trimethylolpropane with hexamethylene diisocyanate trimer.

The content of the crosslinking agent is not particularly limited as long as the living body tacky agent according to this embodiment is formulated so as to satisfy predetermined properties such as gel fraction, and the like. For example, it may be 0.050 parts by mass to 0.20 parts by mass relative to 100 parts by mass of the living body tacky agent.

The living body tacky agent according to this embodiment may be a tacky tape made of the above materials.

The living body tacky agent according to the present embodiment may have a corrugated pattern (web pattern) formed on the surface thereof in such a way that recesses having a thickness less than that of other parts (or having zero thickness) are arranged repeatedly and alternately with the other parts. As the living body tacky agent according to the present embodiment, for example, a tacky tape having a web pattern formed on the surface thereof may be used. The living body tacky agent according to the present embodiment having a web pattern on the surface thereof has both of parts where the tackifier can easily contact the surface of the living body and parts where the tackifier hardly contacts the living body on the surface of the living body tacky agent according to the present embodiment, so that the parts where the tackifier can easily contact the living body can be scattered throughout the surface of the living body tacky agent according to the present embodiment. The moisture permeability of the living body tacky agent according to the present embodiment tends to be higher as the tackifier is thinner. Therefore, by forming a web pattern on the surface of the living body tacky agent according to the present embodiment so as to have parts where the thickness of the tackifier is partially small, it is possible to improve the moisture permeability while maintaining the tacky force, compared to the case where no web pattern is formed. The shape of the recesses may be linear or circular in addition to the corrugated shape.

The thickness of the living body tacky agent according to the present embodiment can be desirably set, and, for example, is preferably from 10 μm to 300 μm and more preferably from 50 μm to 100 μm. When the thickness of a first tacky layer 122 is from 10 μm to 300 μm, the living body tacky agent according to the present embodiment can be reduced in thickness.

As described above, by having a 60° peel tackiness of 5.0 N/cm or greater with respect to an adherend having an Asker C hardness of 0, the living body tacky agent according to this embodiment can match the actual tacky force on the surface of a subject's skin 2 and have a tacky force close to that for actual use. Moreover, the living body tacky agent according to this embodiment can have a tacky force close to that for actual use on the skin 2 regardless of whether the skin 2 is dry, wet with sweat, or wet with water such as a shower. Therefore, the living body tacky agent according to this embodiment can exhibit excellent adhesion reliability on the surface of the skin 2.

### Biosensor

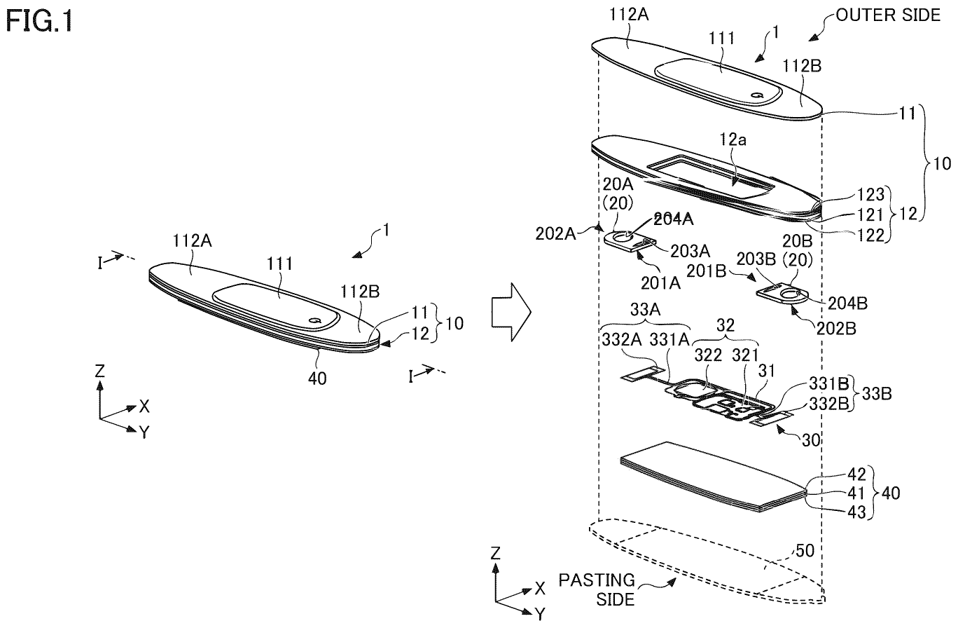

A biosensor to which the living body tacky agent according to this embodiment is applied will be described below. The living body tacky agent according to this embodiment is used on a first tacky layer 122 of an upper sheet 12 of a biosensor 1 described later, as shown in FIG. 1.

In this embodiment, the case where the living body tacky agent according to this embodiment is used on the first tacky layer 122 will be described, but the living body tacky agent according to this embodiment can be used on any tacky layer that is provided at the position where the biosensor 1 contacts a living body. The living body tacky agent according to this embodiment may be used on a second tacky layer 43 of a second layer member 40 of the biosensor 1 described later, or may be used simultaneously on both the first tacky layer 122 of the upper sheet 12 and the second tacky layer 43 of the second layer member 40.

The biosensor to which the living body tacky agent according to this embodiment is applied is a pasting-type biosensor that is pasted on a part of a living body (for example, skin, scalp, forehead, and the like) to measure biological information. In this embodiment, a case where the biosensor is pasted on the skin of a person to measure an electric signal (biological signal) related to biological information of the person will be described.

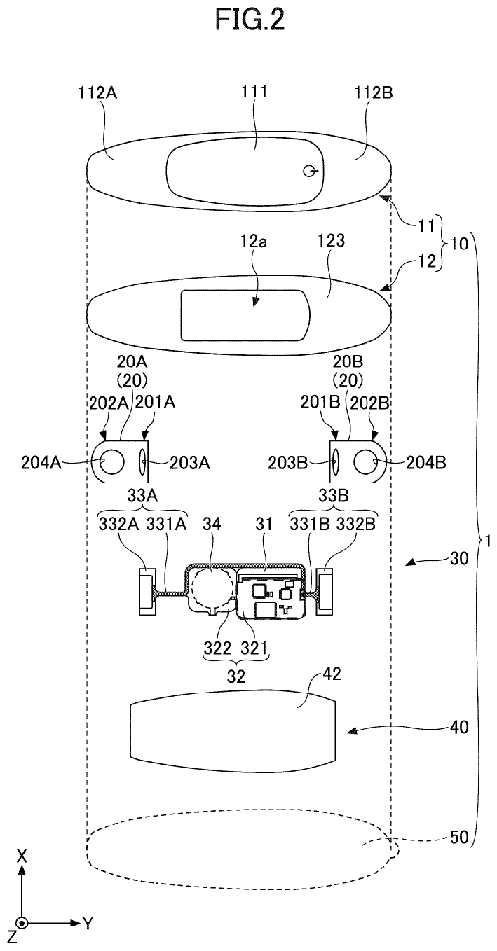

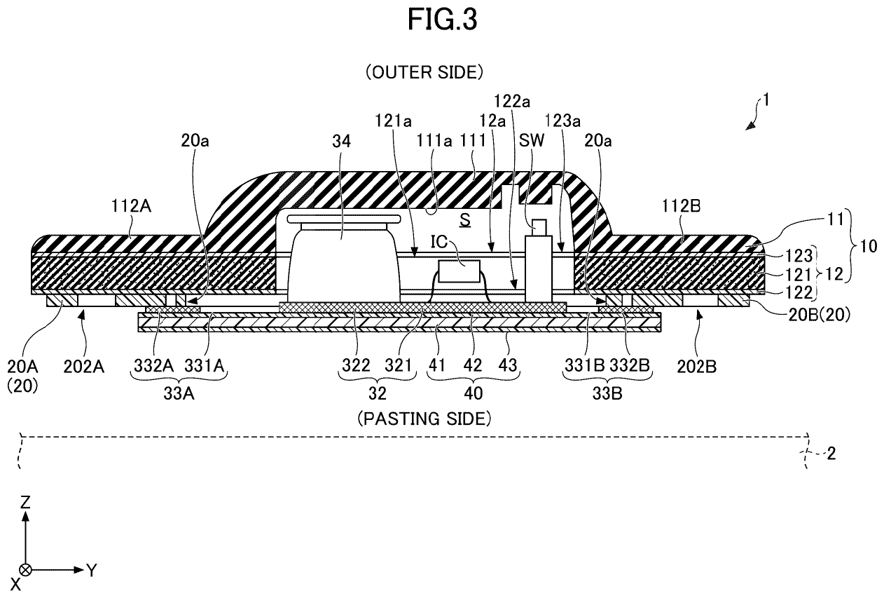

FIG. 1 is an oblique view showing the overall configuration of the biosensor to which the living body tacky agent according to this embodiment is applied. The left side of FIG. 1 shows the appearance of the biosensor, and the right side of FIG. 1 shows the disassembled state of the components of the biosensor. FIG. 2 is a plan view showing an example of the components of the biosensor. FIG. 3 is a longitudinal cross-sectional view of the biosensor and is a cross-sectional view taken along I-I of FIG. 1.

As shown in FIGS. 1 and 2, the biosensor 1 is a plate-like (sheet-like) member formed in a substantially elliptical shape in a plan view. As shown in FIGS. 2 and 3, the biosensor 1 has a first layer member 10, electrodes 20, a sensor part 30, and the second layer member 40, and is formed by laminating the first layer member 10, the electrodes 20, and the second layer member 40 in this order from the first layer member 10 side to the second layer member 40 side. In the biosensor 1, the first layer member 10, the electrodes 20, and the second layer member 40 form a pasting surface to be pasted on skin 2, which is an example of a living body. With the pasting surface pasted on the skin 2, the biosensor 1 measures a potential difference (polarization voltage) between the skin 2 and the electrodes 20, to thereby measure an electric signal (biological signal) related to biological information of the subject.

In FIGS. 1 to 3, using a three-dimensional orthogonal coordinate system in three axial directions (X-axis direction, Y-axis direction, and Z-axis direction), the shorter direction of the biosensor is defined as the X-axis direction, the longer direction thereof is defined as the Y-axis direction, and the height direction (thickness direction) thereof is defined as the Z-axis direction. A direction (outer side) opposite to a side (pasting side) of the biosensor 1 pasted on the living body (subject) is defined as the +Z-axis direction, and the pasting side is defined as the −Z-axis direction. In the following description, for convenience of explanation, the +Z-axis direction may be referred to as the upper side or top, and the −Z-axis direction may be referred to as the lower side or bottom, but this does not represent a universal vertical relationship.

A biological signal is, for example, an electric signal representing an electrocardiogram waveform, a brain wave, a pulse, or the like.

### First Layer Member

As shown in FIGS. 1 and 2, the first layer member 10 includes a cover member 11 and an upper sheet 12 laminated in this order. The cover member 11 and the upper sheet 12 have substantially the same contour shape in a plan view.

In a plan view, the first layer member 10 may have a rectangular shape having a longer direction (Y-axis direction) and a shorter direction (X-axis direction), and may have semi-circularly rounded shapes on both ends in the longer direction.

### Cover Member

As shown in FIG. 3, the cover member 11 is positioned on the outermost side (+Z-axis direction) of the biosensor 1, and is adhesively joined to the upper surface of the upper sheet 12. The cover member 11 has a protrusion 111 that protrudes substantially in a dome shape toward the height direction (+Z-axis direction) of FIG. 1 in the center in the longer direction (Y-axis direction), and flat parts 112A and 112B provided on both ends of the cover member 11 in the longer direction (Y-axis direction). The upper and lower surfaces of the protrusion 111 and the upper and lower surfaces of the flat parts 112A and 112B may be formed flatly.

On the inner side (pasting side) of the protrusion 111, the cover member 11 has an opening formed so as to have a recess 111a formed in a shape concave to the skin 2 side. The recess 111a needs only to have a size that forms at least a part of a storage space S and can store at least a part of the sensor part 30. The storage space S for storing the sensor part 30 is formed on the inner side (pasting side) of the protrusion 111 by the recess 111a on the inner surface of the protrusion 111, the electrodes 20, and the second layer member 40.

The cover member 11 may typically be formed from a flexible material such as crosslinked rubber and the like. Examples of the crosslinked rubber include silicone rubber, fluorine rubber, urethane rubber, natural rubber, acrylic rubber, butadiene rubber, isoprene rubber, styrene-butadiene copolymer rubber, nitrile rubber, hydrogenated nitrile rubber, chloroprene rubber, ethylene-propylene copolymer rubber, chlorinated polyethylene rubber, chlorosulfonated polyethylene rubber, butyl rubber, and halogenated butyl rubber. Using a base resin such as polyethylene terephthalate (PET) as a support, the cover member 11 may be formed by laminating the above flexible material on the surface of the support. With the cover member 11 formed from the above flexible material, the sensor part 30 positioned in the storage space S of the cover member 11 is protected, and any impact applied to the biosensor 1 from the upper surface side is absorbed, to relieve the impact applied to the sensor part 30.

The thickness of the upper surface and sidewall of the protrusion 111 may be larger than the thickness of the flat parts 112A and 112B. This can make the flexibility of the protrusion 111 lower than that of the flat parts 112A and 112B, and can protect the sensor part 30 from external force applied to the biosensor 1.

The thickness of the upper surface and sidewall of the protrusion 111 can be appropriately designed, and may be, for example, 1.5 mm to 3 mm. The thickness of the flat parts 112A and 112B can also be appropriately designed, and may be, for example, 0.5 mm to 1 mm.

Since the flat parts 112A and 112B having a lesser thickness are more flexible than the protrusion 111, when the biosensor 1 is pasted on the skin 2, the flat parts can easily deform following deformation of the surface of the skin 2 due to body movements such as stretching, bending, twisting, and the like. Thus, stress to be applied to the flat parts 112A and 112B when the surface of the skin 2 is deformed can be relaxed. This makes it difficult for the biosensor 1 to be peeled from the skin 2.

The outer periphery of the flat parts 112A and 112B may have a shape gradually decreasing in thickness toward the edge. This can further increase the flexibility of the outer periphery of the flat parts 112A and 112B, and it is possible to improve the feeling of wearing when the biosensor 1 is pasted on the skin 2, compared with the case of not reducing the thickness of the outer periphery of the flat parts 112A and 112B.

The hardness (strength) of the cover member 11 can be appropriately designed to any magnitude, and may be, for example, 40 to 70. When the hardness of the cover member 11 is within the above preferable range, the upper sheet 12, the electrodes 20, and the second layer member 40 can easily deform following the movement of the skin 2 without being affected by the cover member 11 when the skin 2 extends due to a body movement. The hardness refers to Shore A hardness. In this specification, Shore A hardness refers to a value measured in accordance with ISO7619-1 (JIS K 6253-3:2012). The Shore A hardness is a Type A durometer hardness measured by a rubber hardness tester (Type A durometer) using a Type A (cylindrical) indenter. As specified in JIS K 6253-3:2012, “Vulcanized rubber and thermoplastic rubber-How to determine hardness-Part 3: Durometer hardness”, a Type A durometer hardness measured value measured by preparing a sheet sample having a predetermined size using the cover member 11 may be adopted as the Shore A hardness of the cover member 11.

### Upper Sheet

As shown in FIG. 3, the upper sheet 12 is provided being adhesively joined to the lower surface of the cover member 11. The upper sheet 12 contains the living body tacky agent according to the present embodiment. The upper sheet 12 has a through-hole 12a at a position facing the projection 111 of the cover member 11. A sensor body 32 of the sensor part 30 can be stored in the storage space S formed by the recess 111a on the inner surface of the cover member 11 and the through-hole 12a without being interrupted by the upper sheet 12.

The upper sheet 12 includes a first substrate 121, a first tacky layer 122 provided on one surface of the first substrate 121 facing the electrodes 20, such that the electrodes 20 are pasted on the first tacky layer 122, and an upper tacky layer 123 provided on a surface of the first substrate 121 opposite to the one surface facing the electrodes 20.

### First Substrate

As shown in FIG. 3, the first substrate 121 is provided on the pasting side of the cover member 11, which is the opening side. As shown in FIG. 1, the first substrate 121 is formed in a sheet shape. The first substrate 121 may have flexibility, waterproofness, and moisture permeability. With the first substrate 121 having flexibility, waterproofness, and moisture permeability, the first substrate 121 can easily extend while being in a state of having contact with the skin 2, can be maintained in the state of having contact with the skin 2, and can avoid intrusion of a liquid into the gap between the first substrate 121 and the upper tacky layer 123. Moreover, water vapor generated by sweat or the like generated from the skin 2 can be released to the outside of the biosensor 1 through the first substrate 121. This makes it easier for the upper sheet 12 to maintain adhesion durability.

As long as the first substrate 121 has flexibility, waterproofness, and moisture permeability, it may be a non-porous body without a porous structure or a porous body with a porous structure. When the first substrate 121 is a non-porous body, thickness reduction and a sustainable strength of the first substrate 121 are facilitated advantageously. When the first substrate 121 is a porous body, releasing of water vapor generated from sweat or the like generated from the skin 2 on which the biosensor 1 is pasted to the outside of the biosensor 1 through the first substrate 121 is facilitated advantageously.

As the non-porous body, a molded body formed in a sheet shape can be used.

The porous body may have a foamed structure containing, for example, continuous cells, independent cells, semi-independent cells, and the like. That is, the porous body may be a porous body produced by foam molding for forming communicating cells (a porous body having a communicating cell structure), a porous body produced by foam molding for forming independent cells (a porous body having an independent cell structure), or a porous body produced by foam molding for forming semi-independent cells (a porous body having a semi-independent cell structure). Among these, a porous body having an independent cell structure is preferable from the viewpoint of achieving thickness reduction and a sustainable strength while exhibiting higher waterproof properties. For example, a foamed sheet, a nonwoven fabric sheet, or the like can be used as the porous body.

As the material from which the first substrate 121 is formed, a flexible material such as a thermoplastic resin such as a polyurethane resin, a polystyrene resin, a polyolefin resin, a silicone resin, an acrylic resin, a vinyl chloride resin, a polyester resin, or the like, and a thermoplastic elastomer or the like may be used.

Examples of the thermoplastic elastomer include a polyurethane thermoplastic elastomer, a polystyrene thermoplastic elastomer, a polyolefin thermoplastic elastomer, a polyester thermoplastic elastomer, a polyvinyl chloride thermoplastic elastomer, a polyamide thermoplastic elastomer, a nitrile thermoplastic elastomer, a nylon thermoplastic elastomer, a fluororubber thermoplastic elastomer, a polybutadiene thermoplastic elastomer, an ethylene vinyl acetate thermoplastic elastomer, a chlorinated polyethylene thermoplastic elastomer, a styrene-butadiene block copolymer or a hydrogenated product thereof, a styrene-isoprene block copolymer or a hydrogenated product thereof, and the like. One of these may be used alone or two or more of these may be used in combination. Among these, polyurethane thermoplastic elastomer is preferable.

When the first substrate 121 is a non-porous body, specifically, a polyurethane sheet such as Esmer URS available from Nihon Matai Co., Ltd. may be used.

When the first substrate 121 is a porous body, specifically, a foamed sheet such as FOLEC available from Inoac Corporation, or a nonwoven fabric sheet such as a patch medicine base fabric EW available from Japan Vilene Company, Ltd. may be used.

The moisture permeability of the first substrate 121 may be higher than that of the cover member 11. The moisture permeability of the first substrate 121 may be 100 g/(m²·day) to 5,000 g/(m²·day). When the moisture permeability of the first substrate 121 is 100 g/(m²·day) to 5,000 g/(m²·day), the first substrate 121 can allow water vapor intruding from one surface side to pass through the first substrate 121 to be stably released from the other surface side.

The thickness of the first substrate 121 may be appropriately set according to the type of the first substrate 121, and the like, but is preferably larger than the thickness of the flat parts 112A and 112B of the cover member 11. When the thickness of the first substrate 121 is larger than the thickness of the flat parts 112A and 112B of the cover member 11, it is possible to alleviate the skin 2 being irritated by being contacted by the flat parts 112A and 112B of the cover member 11. The thickness of the first substrate 121 may be, for example, 10 μm to 1.5 mm.

When the first substrate 121 is formed of a porous body such as a foamed sheet or a nonwoven fabric sheet, the thickness of the first substrate 121 is preferably, for example, 0.5 mm to 1.5 mm, and more preferably, approximately 1 mm.

When the first substrate 121 is formed of a non-porous body such as a polyurethane sheet, the thickness of the first substrate 121 is preferably, for example, 10 μm to 300 μm, and more preferably, approximately 30 μm.

The first substrate 121 has a through-hole 121a at a position facing the protrusion 111 of the cover member 11. By providing the first tacky layer 122 and the upper tacky layer 123 on the surface of the first substrate 121 other than the through-hole 121a, it is also possible to form through-holes 122a and 123a in the first tacky layer 122 and the upper tacky layer 123. The through-holes 121a, 122a, and 123a form the through-hole 12a.

### First Tacky Layer

As shown in FIG. 3, the first tacky layer 122 is provided in a state of being pasted on one surface of the first substrate 121 facing the electrodes 20. The living body tacky agent according to the present embodiment is used as the first tacky layer 122. As the first tacky layer 122, the living body tacky agent according to this embodiment described above is used.

The first tacky layer 122 is located on the living body side (−Z-axis direction) of the first substrate 121 and has a function of bonding the skin 2 to the first substrate 121, a function of bonding the first substrate 121 to a second substrate 41, and a function of bonding the first substrate 121 to the electrodes 20.

When peeling the first tacky layer 122, which is pasted on an adherend having an Asker C hardness of 0, from the adherend at a peeling angle of 60° with respect to the adherend, the first tacky layer 122 has a peel tackiness of 5.0 N/cm or greater. The peel tackiness is preferably 6.5 N/cm or greater, and more preferably 7.5 N/cm or greater. As the peel tackiness with respect to the adherend at a peeling angle of 60°, the first tacky layer 122 tends to show a value close to the peel tackiness that the first tacky layer 122 possesses when it is actually pasted on the skin 2. Therefore, as long as the first tacky layer 122 has a peel tackiness of 5.0 N/cm or greater at a peeling angle of 60°, it can possess a peel tackiness close to the peel tackiness required when it is actually peeled from the surface of the skin 2 of a subject. Therefore, it can exhibit excellent adhesion reliability to the surface of the skin 2.

The peel tackiness when the peeling angle of the first tacky layer 122 with respect to the adherend having an Asker C hardness of 0 is 60° can be determined by, as described above, pasting the first tacky layer 122 on the human skin gel sheet, and measuring the peel tackiness when subsequently peeling the first tacky layer 122 from the human skin gel sheet at a peeling angle of 60° between the surface of the first tacky layer 122 on the human skin gel sheet side and the human skin gel sheet. As the human skin gel sheet, a commercially available product or the like may be used as described above.

The adhesion reliability with respect to the surface of the skin 2 can be evaluated by performing a tensile endurance test or the like as described above.

As the support, PET or the like may be used as described above.

As described above, the glass transition temperature Tg of the first tacky layer 122 is preferably −57° C. or higher, more preferably −57° C. or higher, and more preferably −45° C. or higher. When the glass transition temperature Tg of the first tacky layer 122 is −57° C. or higher, as described above, the first tacky layer can easily click with an adherend having an Asker C hardness of 0, and can likewise click with and remain pasted on the skin 2 of the subject.

The upper limit of the glass transition temperature Tg of the first tacky layer 122 is not particularly limited, and may be −30° C. or lower. When the glass transition temperature Tg of the first tacky layer 122 is −30° C. or lower, as described above, the first tacky layer 122 can maintain the state of clicking with the adherend having an Asker C hardness of 0, and can therefore likewise click with and be pasted on the skin of the subject as well.

The polymer molecular weight of the first tacky layer 122 can be appropriately selected according to the type of the material forming the first tacky layer 122 and the like as described above, and may be, for example, 500,000 to 1.8 million.

The gel fraction of the first tacky layer 122 is not particularly limited, and may be 25% by mass to 65% by mass, as described above. When the gel fraction is 25% by mass to 65% by mass, the first tacky layer 122 can exhibit excellent peelability and handleability.

The saturated moisture content of the first tacky layer 122 can be appropriately selected according to the type of the material forming the first tacky layer 122 and the like, and may be 0.25% to 0.55% at 22° C. and 50% RH, and may be 0.50% to 1.3% at 40° C. and 92% RH.

The first tacky layer 122 may have moisture permeability as described above. Thus, as will be described later, water vapor due to sweat or the like generated from the skin 2 on which the biosensor 1 is pasted can be dissipated to the first substrate 121 through the first tacky layer 122 and released from the first substrate 121 to the outside of the biosensor 1. When the first substrate 121 has a foamed structure as described above, water vapor can be released to the outside of the biosensor 1 through the first tacky layer 122. As a result, sweat or water vapor can be inhibited from accumulating at the interface between the skin 2 to which the biosensor 1 is attached and the first layer member 10. As a result, it is possible to avoid the biosensor 1 peeling from the skin 2 due to the tacky force of the first tacky layer 122 being weakened by any moisture that was otherwise accumulated at the interface between the skin 2 and the first tacky layer 122.

The moisture permeability of the first tacky layer 122 is preferably, for example, 1 g/(m²·day) or greater. The moisture permeability of the first tacky layer 122 may be 10,000 g/(m²·day) or less. When the moisture permeability of the first tacky layer 122 is 1 g/(m²·day) or greater, sweat or the like propagating through the first tacky layer 122 when the first tacky layer 122 is pasted on the skin 2 can permeate to the outside, thereby reducing the load on the skin 2.

As described above, the material forming the first tacky layer 122 may be a material having a pressure-sensitive adhesiveness, and may contain a polymer as a main component, and may appropriately contain additives such as a tackifier, a liquid component, a crosslinking agent, and the like. Since the material forming the first tacky layer 122 is a material for forming the living body tacky agent according to the present embodiment, details thereof will be omitted.

As described above, the first tacky layer 122 may be a tacky tape formed of the above material.

As described above, the first tacky layer 122 may have a corrugated pattern (web pattern) formed on the surface thereof in such a way that recesses having a thickness less than that of other parts (or having zero thickness) are arranged repeatedly and alternately with the other parts. As the first tacky layer 122, for example, a tacky tape having a web pattern formed on the surface thereof may be used. The first tacky layer 122 having a web pattern on the surface thereof has both of parts where the tackifier can easily contact a living body and parts where the tackifier hardly contacts the living body on the surface of the first tacky layer 122, so that the parts where the tackifier can easily contact the living body can be scattered throughout the surface of the first tacky layer 122. The moisture permeability of the first tacky layer 122 tends to be higher as the first tacky layer 122 is thinner. Therefore, by forming a web pattern on the surface of the first tacky layer 122 so as to have parts where the thickness of the first tacky layer 122 is partially small, it is possible to improve the moisture permeability while maintaining the tacky force, compared with the case where no web pattern is formed.

The thickness of the first tacky layer 122 can be arbitrarily set, and is, for example, preferably from 10 μm to 300 μm, and more preferably from 50 μm to 100 μm. When the thickness of the first tacky layer 122 is 10 μm to 300 μm, the biosensor 1 can be reduced in thickness.

### Upper Tacky Layer

As shown in FIG. 3, the upper tacky layer 123 is provided in a state of being pasted on a surface of the first substrate 121 opposite to the one surface facing the electrodes 20. The upper tacky layer 123 is pasted on the upper surface of the first substrate 121 at positions corresponding to the flat surfaces of the cover member 11 on the pasting side (in the −Z-axis direction), and has a function of bonding the first substrate 121 and the cover member 11.

As the material for forming the upper tacky layer 123, a biocompatible material is used. As the biocompatible material, for example, an acrylic tackifier, a silicone tackifier, a silicone tape, or the like can be used, and it is preferable to use a silicone tackifier.

The thickness of the upper tacky layer 123 can be set appropriately, and may be, for example, 10 μm to 300 μm.

### Electrode

As shown in FIG. 3, the electrodes 20 are pasted on the lower surface of the first tacky layer 122, which is the surface on the pasting side (in the-

Z-axis direction), in a state in which parts of the electrodes 20 on the sensor body 32 side are connected to wires 331A and 331B, and the electrodes are sandwiched between the first tacky layer 122 and a lower tacky layer 42. Parts of the electrodes 20 that are not sandwiched between the first tacky layer 122 and the lower tacky layer 42 contact the living body. When the biosensor 1 is pasted on the skin 2, the electrodes 20 contact the skin 2. This makes it possible to detect a biological signal. The electrodes 20 may be embedded in the second substrate 41 in a state where the electrodes are exposed so that they can contact the skin 2.

The electrodes 20 may be provided under the regions including the flat parts 112A and 112B when viewed in a plan view of the biosensor 1.

The electrodes 20 include a pair of electrodes 20A and 20B. As shown in FIG. 3, the electrode 20A is positioned on the left side in the drawing, and the electrode 20B is positioned on the right side in the drawing. One end side (inner side) of the electrode 20A in the longer direction (Y-axis direction) is in contact with a terminal 332A, and one end (inner side) of the electrode 20B in the longer direction (Y-axis direction) is in contact with a terminal 332B. The pair of electrodes 20A and 20B have substantially the same shape.

The one end side of the electrode 20A that is in contact with the terminal 332A of the sensor part 30 is referred to as a facing part 201A, and the one end side of the electrode 20B that is in contact with the terminal 332B of the sensor part 30 is referred to as a facing part 201B. The other end side (outer side) of the electrode 20A in the longer direction (Y-axis direction), which is a part that is not in contact with the terminal 332A, is referred to as an exposed part 202A, and the other end side (outer side) of the electrode 20B in the longer direction (Y-axis direction), which is a part that is not in contact with the terminal 332B, is referred to as an exposed part 202B.

The electrodes 20 may have any shape such as a sheet shape.

The shape of the electrodes 20 in a plan view is not particularly limited, and may be appropriately designed in any shape according to the application, and the like. As shown in FIG. 2, the electrodes 20A and 20B may, in a plan view, have the facing parts 201A and 201B, which are the one end sides, formed in a rectangular shape, and the exposed parts 202A and 202B, which are the other end sides, formed in an arc shape.

As shown in FIGS. 2 and 3, the electrodes 20A and 20B may have oval through-holes 203A and 203B that are provided at the one end side (inner side) in the longer direction (Y-axis direction) and are elongated in in the width direction (X-axis direction), and circular through-holes 204A and 204B that are provided at the other end side (outer side) in the longer direction (Y-axis direction). As a result, the electrodes 20 can expose the first tacky layer 122 through the through-holes 203A and 203B and the through-holes 204A and 204B to the pasting side while the electrodes are pasted on the first tacky layer 122, so that the close adhesiveness between the electrodes 20 and the skin 2 can be enhanced. The number of through-holes 203A and 203B and the number of through-holes 204A and 204B are not particularly limited, and may be appropriately set according to the size of the facing parts 201A and 201B of the electrodes 20 and the like.

The electrodes 20 can be made of a cured product of a conductive composition containing a conductive polymer and a binder resin, a metal, an alloy, or the like. In particular, it is preferable that the electrodes 20 are made of a cured product of a conductive composition from the viewpoint of the safety of a living body, such as for preventing allergic reactions when the electrodes 20 are applied to the living body. The electrodes 20 may be electrode sheets obtained by forming a cured product of a conductive composition into a sheet shape.

As the conductive polymer, for example, a polythiophene conductive polymer, a polyaniline conductive polymer, a polyacetylene conductive polymer, a polypyrrole conductive polymer, a polyphenylene conductive polymer, derivatives thereof, complexes thereof, and the like can be used. One of these may be used alone, or two or more of these may be used in combination. Among these, it is preferable to use a complex obtained by doping polythiophene with polyaniline as a dopant. Among complexes of polythiophene and polyaniline, it is more preferable to use PEDOT/PSS obtained by doping poly (3,4-ethylenedioxythiophene) (also referred to as PEDOT) serving as polythiophene with polystyrenesulfonic acid (poly 4-styrenesulfonate; PSS) serving as polyaniline, because such a complex has a lower contact impedance with a living body and a high conductivity.

A water-soluble polymer, a water-insoluble polymer, or the like can be used as the binder resin.

As the water-soluble polymer, a hydroxyl group-containing polymer such as polyvinyl alcohol (PVA), modified PVA, or the like can be used.

The conductive composition may contain various commonly used additives such as a crosslinking agent, a plasticizer, and the like at appropriate proportions. Examples of the crosslinking agent include aldehyde compounds such as sodium glyoxylate and the like. Examples of the plasticizer include glycerin, ethylene glycol, propylene glycol, and the like.

Common metals such as Au, Pt, Ag, Cu, Al, and the like, and alloys can be used as the metals and alloys.

The thickness of the electrodes 20 may be any height, and may be, for example, 10 μm to 100 μm. When the thickness of the electrodes 20 is within the above preferred range, the electrodes 20 can have sufficient strength and flexibility, and conductive stability during deformation.

The thickness of the electrodes 20 means the length of the electrodes 20 perpendicular to the surface thereof as in the case of the protrusion 111. The thickness of the electrodes 20 may be measured in the same manner as for the protrusion 111.

The area of the electrodes 20 may be any size depending on the size of the biosensor 1 and the like, and may be, for example, 2.0 cm² to 5.0 cm². If the area of the electrodes 20 is 2.0 cm² to 5.0 cm², the electrodes 20 can have sufficient conductive stability. The method for measuring the area of the electrodes 20 is not particularly limited, and a typical measuring method such as calculation from a plan view image of the electrode may be used.

### Sensor Part

As shown in FIG. 2, the sensor part 30 includes a flexible substrate 31, the sensor body 32, and connection parts 33A and 33B connected to the sensor body 32.

The flexible substrate 31 is a resin substrate on which various components for acquiring biological information are mounted, and the sensor body 32 and connection parts 33A and 33B are placed on the flexible substrate 31.

As shown in FIG. 2, the sensor body 32 includes a component mounting part 321 as a control part and a battery attaching part 322 and is configured to acquire biological information.

The component mounting part 321 includes various components that are mounted on the flexible substrate 31, such as a CPU and an integrated circuit for processing biological signals acquired from a living body to generate biological signal data, a switch SW for starting the biosensor 1, a flash memory that stores biological signals, a light emitting element, and the like, and is configured to acquire biological information. Circuit examples composed of the various components are omitted. The component mounting part 321 operates based on power supplied from a battery 34 attached to the battery attaching part 322.

The component mounting part 321 performs wired or wireless transmission to an external device such as an operation check device for checking an initial operation, a reading device for reading biological information from the biosensor 1, and the like.

The battery attaching part 322 is situated between the connection part 33A and the component mounting part 321 and supplies power to the integrated circuit and the like mounted on the component mounting part 321. As shown in FIG. 2, the battery 34 is attached to the battery attaching part 322.

The connection parts 33A and 33B have the wires 331A and 331B that are connected to the sensor body 32 in the longer direction (Y-axis direction) of the sensor body 32, and the terminals 332A and 332B that are provided at ends of the wires 331A and 331B and are connected to the electrodes 20.

As shown in FIG. 3, the ends of the wires 331A and 331B on one side are connected to the electrodes 20. As shown in FIG. 3, the end of the wire 331A on the other side is connected to the switch SW and the like mounted on the component mounting part 321 along the outer periphery of the sensor body 32. The end of the wire 331B on the other side is connected to the switch SW and the like mounted on the component mounting part 321.

The terminals 332A and 332B are situated in a state of being sandwiched between the first layer member 10 and the second layer member 40 while one end of each terminal 332A and 332B on one side is connected to the wires 331A or 331B and the upper surfaces of the ends thereof on the other side are in contact with the respective electrodes 20.

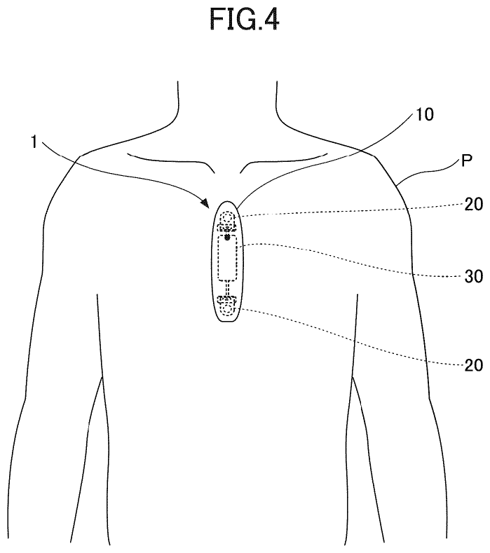

As shown in FIG. 4, the connection parts 33A and 33B may be formed under the flat parts 112A and 112B when viewed in a plan view of the biosensor 1.

A publicly-known battery may be used as the battery 34. A coin type battery such as CR2025 or the like may be used as the battery 34.

### Second Layer Member

As shown in FIG. 3, the second layer member 40 is provided on the pasting surface side of the electrodes 20 and the sensor part 30, serves as a supporting substrate on which the sensor part 30 is installed, and forms a part of the pasting surface to be pasted on the skin 2. As shown in FIGS. 1 and 2, the contour shape of both sides of the second layer member 40 in the width direction (X-axis direction) may be substantially the same as the contour shape of both sides of the first layer member 10 in the width direction (X-axis direction). The length (Y-axis direction) of the second layer member 40 is shorter than the length (Y-axis direction) of the cover member 11 and the upper sheet 12. As shown in FIG. 3, both ends of the second layer member 40 in the longer direction are at positions to sandwich the wires 331A and 331B of the sensor part 30 between the second layer member 40 and the upper sheet 12, and to overlap with part of the electrodes 20.

The second layer member 40 includes the second substrate 41, the lower tacky layer 42 provided on the upper surface of the second substrate 41, and a second tacky layer 43 provided on the lower surface of the second substrate 41. The second substrate 41, the lower tacky layer 42, and the second tacky layer 43 may have the same shape when viewed in a plan view. The second tacky layer 43 of the second layer member 40 and the electrodes 20 form the pasting surface to be pasted on the skin 2. It is possible to vary waterproofness and moisture permeability, and to vary tackiness depending on the position on the pasting surface that depends on the areas of the electrodes 20 and the second tacky layer 43, so that it is possible to vary waterproofness and moisture permeability and to also vary tackiness depending on the area of the pasting surface of the second tacky layer 43.

### Second Substrate

The second substrate 41 can be formed using a flexible resin having appropriate elasticity, flexibility, and toughness. As the material for forming the second substrate 41, for example, it is possible to use: polyester resin such as polyethylene terephthalate (PET), polybutylene terephthalate, polytrimethylene terephthalate, polyethylene naphthalate, and polybutylene naphthalate; acrylic resins such as polyacrylic acid, polymethacrylic acid, polymethyl acrylate, polymethyl methacrylate (PMMA), poly(ethyl methacrylate), poly(butyl acrylate), and the like; polyolefin resins such as polyethylene, polypropylene, and the like; polystyrene resins such as polystyrene, imide-modified polystyrene, acrylonitrile-butadiene-styrene (ABS) resin, imide-modified ABS resin, styrene-acrylonitrile copolymer (SAN) resin, acrylonitrile-ethylene-propylene-diene-styrene (AES) resin, and the like; polyimide resins; polyurethane resins; silicone resins; and thermoplastic resins such as polyvinyl chloride resins such as polyvinyl chloride, vinyl chloride-vinyl acetate copolymer resins, and the like. Among them, polyolefin resins and PET are suitably used. These thermoplastic resins have waterproof properties that do not allow permeation of moisture and water vapor (low moisture permeability). Therefore, by being formed from any of these thermoplastic resins, the second substrate 41 can inhibit sweat or water vapor generated from the skin 2 from intruding into the flexible substrate 31 side of the sensor part 30 through the second substrate 41 when the biosensor 1 is pasted on the skin 2 of the living body.

It is preferable that the second substrate 41 is formed in a flat plate shape because the sensor part 30 is installed on the upper surface thereof via the lower tacky layer 42.

The thickness of the second substrate 41 can be appropriately and desirably selected, and may be, for example, 1 μm to 300 μm.

### Lower Tacky Layer

As shown in FIG. 3, the lower tacky layer 42 is disposed on the upper surface of the second substrate 41 on the cover member 11 side (in +Z-axis direction), and the sensor part 30 is attached to it. Both ends, in the longer direction, of the lower tacky layer 42 of the second layer member 40 are provided at positions facing the facing parts 201A and 201B of the electrodes 20. Thus, the facing parts 201A and 201B of the electrodes 20 and the terminals 332A and 332B can be sandwiched, in a pressed state, between the upper sheet 12 and the second layer member 40, such that the electrodes 20 can be electrically connected with the terminals 332A and 332B. Since the lower tacky layer 42 can be made of the same material as that of the second tacky layer 43 described later, details thereof will be omitted. The lower tacky layer 42 does not necessarily need to be provided, and does not need to be provided.

### Second Tacky Layer

As shown in FIG. 3, the second tacky layer 43 is a layer that is provided on the lower surface of the second substrate 41 on the pasting side (in the −Z-axis direction) and is to be in contact with a living body.

It is preferable that the second tacky layer 43 has pressure-sensitive adhesiveness. Due to the second tacky layer 43 having a pressure-sensitive adhesiveness, it is possible to easily paste the biosensor 1 on the skin 2 of a living body by pressing the biosensor against the skin 2.

The material of the second tacky layer 43 is not particularly limited as long as it is a material having pressure-sensitive adhesiveness, and examples of the material include materials having biocompatibility. Examples of the material for forming the second tacky layer 43 include an acrylic pressure-sensitive adhesive, a silicone pressure-sensitive adhesive, and the like. A preferable example of the material is an acrylic pressure-sensitive adhesive.

It is preferable that the acrylic pressure-sensitive adhesive contains an acrylic polymer as a main component. The acrylic polymer can function as a pressure-sensitive adhesive component. As the acrylic polymer, a polymer obtained by polymerizing a monomer component containing a (meth) acrylate ester such as isononyl acrylate, methoxyethyl acrylate, or the like as a main component and containing a monomer copolymerizable with the (meth) acrylate ester, such as acrylic acid, as an optional component can be used.

It is preferable that the acrylic pressure-sensitive adhesive further contains a carboxylate ester. The carboxylate ester functions as a pressure-sensitive adhesive force regulator that reduces the pressure-sensitive adhesive force of the acrylic polymer and regulates the pressure-sensitive adhesive force of the second tacky layer 43. As the carboxylate ester, a carboxyl ester compatible with the acrylic polymer can be used. As the carboxylate ester, triglyceryl fatty acid ester or the like can be used. The acrylic pressure-sensitive adhesive may contain a crosslinking agent as needed. The crosslinking agent is a crosslinking component that crosslinks the acrylic polymer. Examples of the crosslinking agent include polyisocyanate compounds (polyfunctional isocyanate compounds), epoxy compounds, melamine compounds, peroxide compounds, urea compounds, metal alkoxide compounds, metal chelate compounds, metal salt compounds, carbodiimide compounds, oxazoline compounds, aziridine compounds, amine compounds, and the like. Among these, polyisocyanate compounds are preferable. These crosslinking agents may be used alone or in combination.

It is preferable that the second tacky layer 43 has excellent biocompatibility. For example, when the second tacky layer 43 is subjected to the exfoliation test, it is preferable that the exfoliation area ratio is 0% to 50%. When the exfoliation area ratio is within the range of 0% to 50%, pasting the second tacky layer 43 on the skin 2 can avoid loading the skin 2.

It is preferable that the second tacky layer 43 has moisture permeability. Water vapor or the like generated from the skin 2 on which the biosensor 1 is pasted can be released to the upper sheet 12 side through the second tacky layer 43. Since the upper sheet 12 has a foamed structure as described later, water vapor can be released to the outside of the biosensor 1 through the second tacky layer 43. As a result, sweat or water vapor can be inhibited from accumulating at the interface between the skin 2 to which the biosensor 1 is attached and the second tacky layer 43. As a result, it is possible to avoid the biosensor 1 peeling from the skin due to the tacky force of the second tacky layer 43 being weakened by any moisture that were otherwise accumulated at the interface between the skin 2 and the second tacky layer 43.

The moisture permeability of the second tacky layer 43 may be, for example, 300 g/(m²·day) to 10,000 g/(m²·day). When the moisture permeability of the second tacky layer 43 is within the above range, sweat or the like generated from the skin 2 when the second tacky layer 43 is pasted on the skin 2 can appropriately permeate the second tacky layer 43 to the outside, thereby reducing the load on the skin 2.

The thickness of the second tacky layer 43 can be desirably selected, and may be 10 μm to 300 μm. When the thickness of the second tacky layer 43 is within the above range, the biosensor 1 can be reduced in thickness.

As shown in FIGS. 1 and 2, when the biosensor 1 is not in use, it is preferable to paste a release liner 50 to the pasting surface of the electrodes 20 and the second substrate 41 with the living body until use in order to protect the electrodes 20 and the second layer member 40. When in use, the release liner 50 can be peeled from the electrodes 20 and the second layer member 40, and the pasting surface of the biosensor 1 can be pasted on the skin 2. By pasting the release liner 50 on the pasting surface, it is possible to maintain the tackiness of the electrodes 20 and the second layer member 40 even through a long term of storage or the like of the biosensor 1. Therefore, when in use, by peeling the release liner 50 from the second layer member 40 and the electrodes 20, it is possible to use the sensor by reliably pasting the pasting surface on the skin 2.

The method for manufacturing the biosensor 1 is not particularly limited and any method may be used for the manufacture. An example of the method for manufacturing the biosensor 1 will be described below. The first layer member 10, the electrodes 20, the sensor part 30, and the second layer member 40 shown in FIGS. 1 and 2 are prepared. The first layer member 10, the electrodes 20, the sensor part 30, and the second layer member 40 can be manufactured by any appropriate manufacturing method, as long as the method can manufacture them.

After the first layer member 10, the electrodes 20, the sensor part 30, and the second layer member 40, which are constituents of the biosensor 1 shown in FIG. 1, are prepared, the sensor part 30 is installed on the second layer member 40. Then, the first layer member 10, the electrodes 20, the sensor part 30, and the second layer member 40 are laminated in this order, from the first layer member 10 side to the second layer member 40 side. Thus, the biosensor 1 shown in FIG. 1 is obtained.

FIG. 4 is a diagram showing a state in which the biosensor 1 of FIG. 1 is pasted on the chest of the subject P. As shown in FIG. 4, for example, the biosensor 1 is pasted on the skin of the subject P with the longer direction (Y-axis direction) aligned with the sternum of the subject P, and with one electrode 20 positioned on the upper side and the other electrode 20 positioned on the lower side. The biosensor 1 acquires a biological signal such as an electrocardiogram signal from the subject P by the electrodes 20, with the electrodes 20 brought into pressure contact with the skin of the subject P by the pasting of the biosensor on the skin of the subject P with the second tacky layer 43 of FIG. 2. The biosensor 1 stores the acquired biological signal data in a nonvolatile memory such as a flash memory mounted on the component mounting part 321.

Thus, the biosensor 1 includes the first layer member 10, the electrodes 20, and the sensor body 32, and the first layer member 10 may have the upper sheet 12. Since the upper sheet 12 has the living body tacky agent according to this embodiment and has a tacky force close to that for actual use, it can exhibit excellent adhesion reliability to the surface of the skin 2. The biosensor 1 can have the upper sheet 12 stably pasted on the skin 2, and have the electrodes 20 contact the surface of the skin 2 while being pasted on the first layer member 10. Therefore, the biosensor 1 can be more stably pasted on the skin 2, and reduce the contact impedance of the electrodes 20 with the surface of the skin 2. Therefore, the biosensor 1 can stably maintain a pasting property on the skin 2 during use, and improve measurement accuracy by reducing noise contamination of biological signals.

In the biosensor 1, the upper sheet 12, which is a constituent of the first layer member 10, has the first substrate 121 and the first tacky layer 122, and the first tacky layer 122 is formed of the living body tacky agent according to the present embodiment described above. Therefore, the biosensor 1 can have the first tacky layer 122 stably pasted on the skin 2, and have the electrodes 20 contact the surface of the skin 2 in a state of being pasted on the first layer member 10 by the first tacky layer 122. Therefore, the biosensor 1 can be more stably pasted on the skin 2, and reduce the contact impedance of the electrodes 20 with the surface of the skin 2.

The biosensor 1 has the cover member 11 and the upper tacky layer 123 in the first layer member 10, and the first substrate 121 of the upper sheet 12 can have the through-hole 121a. Since the first tacky layer 122 has tackiness, the electrodes 20 can be brought into contact with the surface of the skin 2 in a state of being stably pasted on the first layer member 10 by the first tacky layer 122. Thus, the biosensor 1 can maintain the electrodes 20 in a state of being stably pasted on the skin 2, and inhibit their positional shift even when the body moves, so the electrodes 20 can reduce the contact impedance with the surface of the skin 2 and avoid noise generation, and can be more stably pasted on the skin 2. Furthermore, since the first substrate 121 has a shape matching the outermost contour shape of the cover member 11, it is possible to prevent the skin 2 from being contacted only by the cover member 11. Therefore, the biosensor 1 can improve the detection accuracy of biological signals and maintain the pasting property on the skin 2, and can reduce the discomfort that may be caused by contact of the outer periphery of the cover member 11 with the subject during use.

The biosensor 1 includes the second layer member 40 and can have the second tacky layer 43 on the surface of the second layer member 40 opposite to the first layer member 10 side. Thus, the biosensor 1 can have the second layer member 40 pasted on the skin 2 via the second tacky layer 43, and therefore, the contact impedance of the electrodes 20 with the surface of the skin 2 can be reduced. Therefore, the biosensor 1 can further enhance the detection accuracy of biological signals during use, and maintain the pasting property on the skin 2 more stably.

The pasting surface of the biosensor 1 on the skin 2 can be formed by the first layer member 10, the electrodes 20, and the second layer member 40. Thus, the biosensor 1 can be reduced in thickness.

Therefore, the biosensor 1 can be smaller in size, and reduce the contact impedance with the surface of the skin 2.

In the biosensor 1, the second tacky layer 43 of the second layer member 40 can be formed of the living body tacky agent according to the present embodiment described above. Since the second tacky layer 43 forms the pasting surface on the skin 2 like the first tacky layer 122, the biosensor 1 can have the second tacky layer 43 stably pasted on the skin 2.

In the biosensor 1, the first tacky layer 122 and the second tacky layer 43 can be formed of the living body tacky agent according to the present embodiment described above. The first tacky layer 122 and the second tacky layer 43 form the entirety of the pasting surface of the biosensor 1 on the skin 2.

Therefore, since the entirety of the pasting surface of the biosensor 1 on the skin 2 can be formed of the living body tacky agent according to the present embodiment described above, the biosensor 1 can enhance the pasting property on the skin 2 and be more stably pasted on the skin 2.

As described above, the biosensor 1 can stably measure biological information from the skin 2 for a long time during use, so it can be effectively used as a pasting-type biosensor to be used while being pasted on the skin 2 of a person or the like. For example, the biosensor 1 can be suitably used as a wearable device for healthcare or the like that is pasted on the skin of a living body or the like, has a high measurement sensitivity of an electrocardiogram, and is required to have a high control effect on noise that may be generated in an electrocardiogram.

Although the embodiments have been described, the above-described embodiments are presented as examples, and the present invention is not limited by the above-described embodiments. The above embodiments can be implemented in various other forms, and various combinations, omissions, substitutions, modifications, and the like are applicable without departing from the spirit of the invention. These embodiments and modifications thereof are included in the scope and spirit of the invention, and are also included in the scope of the invention described in the claims and equivalents thereof.

### EXAMPLES

The embodiments will be described more specifically by presenting examples and comparative examples, but the embodiments are not limited by these examples and comparative examples.

### Production of Acrylic Polymer

### Production of Acrylic Polymer (Polymer Component A)

A polymer (hereinafter referred to as “polymer component A”) containing isononyl acrylate, 2-methoxyethyl acrylate, and acrylic acid at a ratio of 65 parts by mass: 30 parts by mass: 5 parts by mass was produced as an acrylic polymer.

### Production of Acrylic Polymer (Polymer Component B)

A polymer (hereinafter referred to as “polymer component B”) containing acrylic acid-2 ethylhexyl, 1-vinyl-2-pyrrolidone, acrylic acid, and 4-hydroxybutyl acrylate at a ratio of 89.5 parts by mass: 6.0 parts by mass: 4.0 parts by mass: 0.5 parts by mass was produced as an acrylic polymer.

### Production of Acrylic Polymer (Polymer Component C)

A polymer (hereinafter referred to as “polymer component C”, a high molecular weight type of the component A) containing isononyl acrylate, 2-methoxyethyl acrylate, and acrylic acid at a ratio of 65 parts by mass: 30.0 parts by mass: 5 parts by mass was prepared as an acrylic polymer.

### Production of Acrylic Polymer (Polymer Component D)

A polymer (hereinafter referred to as “polymer component D”) containing 2-ethylhexyl acrylate and acrylic acid at a ratio of 95 parts by mass: 5 parts by mass was produced as an acrylic polymer.

### Production of Living Body Tacky Agent

### Production of Living Body Tacky Agent 1

A tackifier composition was produced by mixing 100 parts by mass of the polymer component A as a polymer, 20 parts by mass of a tackifier (KE-311, obtained from Arakawa Chemical Industries, Ltd.), and 0.075 parts by mass of a crosslinking agent (Coronate HL, obtained from Nippon Polyurethane Industry Co., Ltd.). The tackifier composition was applied, to produce a living body tacky agent 1 (having a length of 200 mm, a width of 100 mm, and a thickness of 60 μm).

The living body tacky agent 1 had a polymer molecular weight of 700,000 to 800,000, a gel fraction of 41.8%, a glass transition temperature Tg of −40.4° C., and a saturated moisture content of 0.50% at 22° C. and RH 50%, and 1.11% at 40° C. and RH 92%.

### Production of Living Body Tacky Agent 2

A tackifier composition was produced by mixing 100 parts by mass of the polymer component B as a polymer and 0.1 parts by mass of a crosslinking agent (Coronate L, obtained from Nippon Polyurethane Industry Co., Ltd.). The tackifier composition was applied, to produce a living body tacky agent 2 (having a length of 200 mm, a width of 100 mm, and a thickness of 30 pm). The living body tacky agent 2 had a polymer molecular weight of 800,000 to 900,000, a gel fraction of 29.8%, a glass transition temperature Tg of −44.7° C., and a saturated moisture content of 0.33% at 22° C. and RH 50%, and 0.72% at 40° C. and RH 92%.

### Production of Living Body Tacky Agent 3

A tackifier composition was produced by mixing 100 parts by mass of the polymer component A as a polymer, 10 parts by mass of a tackifier (Haritack PCJ, obtained from Harima Chemicals, Inc.), 15 parts by mass of a liquid component (Coconade RK, obtained from Kao Corporation), and 0.075 parts by mass of a crosslinking agent (Coronate HL, obtained from Nippon Polyurethane Industry Co., Ltd.). The tackifier composition was applied, to produce a living body tacky agent 3 (having a length of 200 mm, a width of 100 mm, and a thickness of 60 μm). The living body tacky agent 3 had a polymer molecular weight of 700,000 to 800,000, a gel fraction of 37.5%, a glass transition temperature Tg of −54.5° C., and a saturated moisture content of 0.52% at 22° C. and RH 50%, and 1.08% at 40° C. and RH 92%.

### Production of Living Body Tacky Agent 4

A tackifier composition was produced by mixing 100 parts by mass of the polymer component C as a polymer, 10 parts by mass of a tackifier (Haritack PCJ, obtained from Harima Chemicals, Inc.), 15 parts by mass of a liquid component (Coconade RK, obtained from Kao Corporation), and 0.075 parts by mass of a crosslinking agent (Coronate HL, obtained from Nippon Polyurethane Industry Co., Ltd.). The tackifier composition was applied, to produce a living body tacky agent 4 (having a length of 200 mm, a width of 100 mm, and a thickness of 60 μm). The living body tacky agent 4 had a polymer molecular weight of approximately 1.8 million, a gel fraction of 60.6%, a glass transition temperature Tg of −35.6° C., and a saturated moisture content of 0.51% at 22° C. and RH 50%, and 0.98% at 40° C. and RH 92%.

### Production of Living Body Tacky Agent 5

A tackifier composition was produced by mixing 100 parts by mass of the polymer component D as a polymer and 5 parts by mass of a tackifier (KE-311, obtained from Arakawa Chemical Industries, Ltd.). The tackifier composition was applied, to produce a living body tacky agent 5 (having a length of 200 mm, a width of 100 mm, and a thickness of 40 μm). The living body tacky agent 5 had a polymer molecular weight of 1.1million to 1.3 million, a gel fraction of 6.5%, a glass transition temperature Tg of −56.2° C., and a saturated moisture content of 0.27% at 22° C. and RH 50%, and 0.55% at 40° C. and RH 92%.

### Production of Living Body Tacky Agent 6

A tackifier composition composed of 100 parts by mass of the polymer component D as a polymer was produced. The tackifier composition was applied, to produce a living body tacky agent 6 (having a length of 200 mm, a width of 100 mm, and a thickness of 40 μm). The living body tacky agent 6 had a polymer molecular weight of 1.1 million to 1.3 million, a gel fraction of 3.9%, a glass transition temperature Tg of −59.0° C., and a saturated moisture content of 0.22% at 22° C. and RH 50%, and 0.45% at 40° C. and RH 92%.

### Measurement of 60° Peel Tackiness

Each of the living body tacky agents 1 to 6 described above was laminated on one main surface of a rectangular support (PET, having a thickness of 25 μm), to produce a laminate (living body tacky member). Each laminate thus produced was pasted on a human skin gel sheet (H0-1, obtained from Exseal Corporation). Then, the tacky force (60° peel tackiness) was measured at peeling of the laminate, starting from one longer-direction end of the laminate, from the human skin gel sheet at normal temperature and normal humidity (22° C., 50 RH %) at a peeling angle of 60° between a surface of each laminate on the human skin gel sheet side and the human skin gel sheet at a peeling speed of 150 mm/min. The human skin gel sheet had been stored at normal temperature and normal humidity (22° C., 50RH %) for 3 days in order to stabilize the internal moisture content. The moisture content in the human skin gel sheet after being stored at normal temperature and normal humidity for 3 days was approximately 0.65%.

### Measurement of 60° Peel Tackiness When Using a Human Skin Gel Sheet Stored at High Temperature and High Humidity

A human skin gel sheet stored at high temperature and high humidity (60° C., 90 RH %) for 3 days was used instead of a human skin gel sheet stored at normal temperature and normal humidity (22° C., 50 RH %) for 3 days. The moisture content in the human skin gel sheet after being stored at high temperature and high humidity for 3 days was approximately 1.89%. By storing the human skin gel sheet at high temperature and high humidity and increasing the moisture content, a state similar to skin damp with sweat or skin wet with water was simulated, and the 60° peel tackiness was measured. After storing the human skin gel sheet at high temperature and high humidity for 3 days, each living body tacky agent was pasted on the human skin gel sheet at high temperature and high humidity (60° C., 90 RH %) and left to stand for 10 minutes to 30 minutes. Then, the 60° peel tackiness was measured in the same manner as described above.

### Measurement of 180° Peel Tackiness

After pasting each of the laminates of the living body tacky agents 1 to 6 on a bakelite plate (a phenol laminate plate, obtained from Standard Testpiece Co., Ltd.), the tacky force (180° peel tackiness) was measured at peeling of the laminate, starting from one longer-direction end of the laminate, from the bakelite plate at a peeling angle of 180° between a surface of each laminate on the bakelite plate side and the bakelite plate.

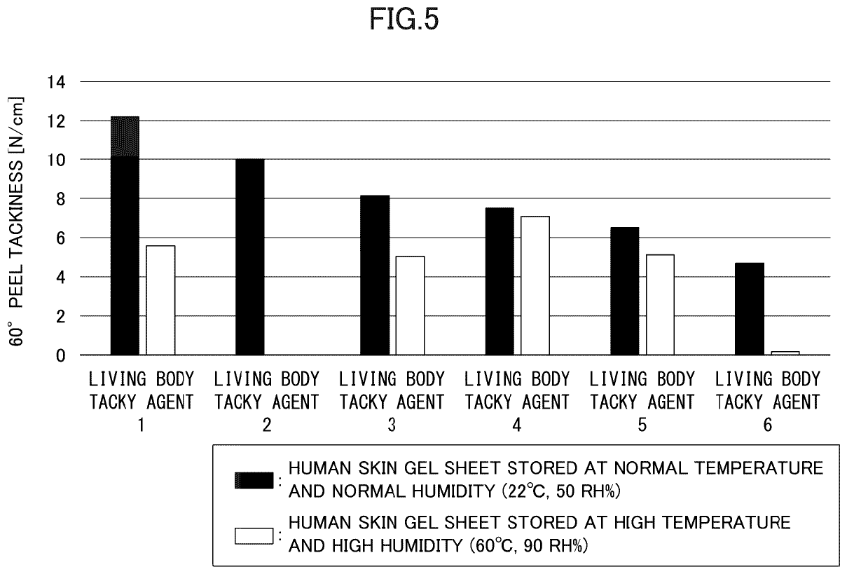

The thickness, the composition, the polymer molecular weight, the gel fraction, the glass transition temperature Tg, and the saturated moisture content of each living body tacky agent, and the measurement results of the 60° peel tackiness and the 180° peel tackiness of each living body tacky agent laminate are indicated in Table 1. When the adherend was the human skin gel sheet, measurement results obtained under the condition that the adherend was stored at normal temperature and normal humidity (22° C., 50 RH %) and high temperature and high humidity (60° C., 90 RH %) for 3 day are indicated as the 60° peel tackiness. The measurement results of the 60° peel tackiness of the laminates when the human skin gel sheet stored at normal temperature and normal humidity (22° C., 50 RH %) and at high temperature and high humidity (60° C., 90 RH %) was used as the adherend are indicated in FIG. 5.

TABLE 1

| | | | L.B. | L.B. | L.B. | L.B. | L.B. | L.B. |
|---|---|---|---|---|---|---|---|---|
| | | | Tac. Agt. | Tac. Agt. | Tac. Agt. | Tac. Agt. | Tac. Agt. | Tac. Agt. |
| | | | 1 | 2 | 3 | 4 | 5 | 6 |
| Thickness [μm] | | | 60 | 30 | 60 | 60 | 40 | 40 |
| Compos. | Polymer | | Polymer | Polymer | Polymer | Polymer | Polymer | Polymer |
| [pts. | | | comp. | comp. | comp. | comp. | comp. | comp. |
| mass] | | | A (100) | B (100) | A (100) | C (100) | D (100) | D (100) |
| | Tackifier | | KE-311 (20) | — | Haritack | Haritack | KE-311 (5) | — |
| | | | | | PCJ (10) | PCJ (10) | | |
| | Liquid component | | — | — | Coconade | Coconade | — | — |
| | | | | | RK (15) | RK (15) | | |
| | Crosslinking agent | | Coronate | Coronate | Coronate | Coronate | — | — |
| | | | HL (0.075) | L (0.1) | HL (0.075) | HL (0.075) | | |
| Polymer molecular weight MW | | | 700,000 to | 800,000 to | 700,000 to | Approx. 180 | 110 to 130 | 110 to 130 |
| | | | 800,000 | 900,000 | 800,000 | million | million | million |
| Gel fraction [mass %] | | | 41.8 | 29.8 | 37.5 | 60.6 | 6.5 | 3.9 |
| Tg [° C.] | | | −40.4 | −44.7 | −54.5 | −35.6 | −56.2 | −59 |
| Satd. | 22° C., 50% | | 0.5 | 0.33 | 0.52 | 0.51 | 0.27 | 0.22 |
| moisture | 40° C. 92% | | 1.11 | 0.72 | 1.08 | 0.98 | 0.55 | 0.45 |
| content | | | | | | | | |
| [%] | | | | | | | | |
| 60° peel | adherend | HS gel | 12.20 | 10.00 | 8.14 | 7.50 | 6.50 | 4.69 |
| tac. | | sheet | | | | | | |
| [N/cm] | | (Asker C | | | | | | |
| | | HR: 0 | | | | | | |
| | | (stored | | | | | | |
| | | at 22° C., | | | | | | |
| | | 50RH %, 3 | | | | | | |
| | | days) | | | | | | |
| | | HS gel | 5.58 | — | 5.04 | 7.07 | 5.12 | 0.18 |
| | | sheet | | | | | | |
| | | (Asker C | | | | | | |
| | | HR: 0 | | | | | | |
| | | (stored | | | | | | |
| | | at 60° C., | | | | | | |
| | | 90RH %, 3 | | | | | | |
| | | days) | | | | | | |
| 180° | | Bakelite | 6.22 | 8.20 | 3.73 | — | — | 4.83 |
| peel | | pl. | | | | | | |
| tac. | | (stored | | | | | | |
| [N/cm] | | at 22° C., | | | | | | |
| | | 50RH %, 3 | | | | | | |
| | | days) | | | | | | |

### Production of Biosensor

### Example 1

### Production of Cover Member

A cover member was produced by forming a coating layer having a shore hardness of A40 and made of silicone rubber on a support formed using PET as a base resin, and molding them into a predetermined shape.

### Production of First Laminate Sheet

A first tacky layer was formed by pasting the living body tacky agent 1 on the lower surface of a first substrate formed in a rectangular shape (FOLEC (registered trademark), obtained from INOAC Corporation, having a thickness of 1 mm). Then, a silicone tape (having a thickness of 60 μm) was pasted on the upper surface of the pasting layer to form an upper tacky layer, to thereby produce a first laminate sheet.

### Production of Electrodes

### 1. Production of Conductive Composition

Zero point thirty-eight parts by mass of a PEDOT/PSS pellet (Orgacon DRY, obtained from AGFA Materials Japan) as a conductive polymer, 10.00 parts by mass of a modified polyvinyl alcohol (modified PVA)-containing aqueous solution (having a modified polyvinyl alcohol concentration of 10%, Gohsenx Z-410, obtained from Nippon Synthetic Chemical Industry Co., Ltd.) as a binder resin, 2.00 parts by mass of glycerin (obtained from Wako Pure Chemical Industries, Ltd.) as a plasticizer, and 1.60 parts by mass of 2-propanol and 6.50 parts by mass of water as solvents were added into an ultrasonic bath. Then, the aqueous solution containing these components was mixed for 30 minutes in the ultrasonic bath, to prepare a homogeneous conductive composition aqueous solution A.

Since the modified PVA concentration in the modified PVA-containing aqueous solution was approximately 10%, the content of the modified PVA in the conductive composition aqueous solution A was 1.00 part by mass. The balance was the solvent of the conductive composition aqueous solution A.

The contents of the conductive polymer, the binder resin, and the plasticizer relative to 100.0 parts by mass of the conductive composition were 11.2 parts by mass, 29.6 parts by mass, and 59.2 parts by mass, respectively.

### 2. Production of Electrode Sheet

The prepared aqueous solution A of the conductive composition was coated on a polyethylene terephthalate (PET) film using an applicator. Then, the PET film on which the aqueous solution A of the conductive composition was coated was transported to a drying oven (SPHH-201, ESPEC), and the aqueous solution A of the conductive composition was heated and dried at 135° C. for 3 minutes to prepare a cured product of the conductive composition. The cured product was punched (pressed) into a desired shape to form a sheet, and an electrode, which is an electrode sheet (bioelectrode) with a thickness of 20 μm, was prepared.

The contents of the conductive polymer, binder resin, and plasticizer contained in the electrode sheet were the same as those of the conductive composition, and were 11.2 parts by mass, 29.6 parts by mass, and 59.2 parts by mass, respectively.

### Production of Second Laminate Sheet

A tackifier (PERMEROLL, obtained from Nitto Denko Corporation, having a moisture permeability of 21 g/(m²·day)) was pasted on both sides of a second substrate formed in a rectangular shape (PET (PET-50 SCA1 (white), obtained from Mitsui & Co. Plastics, Ltd.), having a thickness of 38 μm) to form a lower tacky layer and a second tacky layer, to thereby produce a second laminate sheet.

### Production of Biosensor

A sensor part equipped with a battery and a control part was installed in the center of the upper surface of the second laminate sheet. Then, while being sandwiched between the first tacky layer of the first laminate sheet and the second laminate sheet, a pair of electrodes were pasted on the pasting surface side of the first tacky layer. The electrodes and the wires of the sensor part were connected. Then, the cover member was laminated on the first laminate sheet such that the sensor part would be positioned within the storage space formed by the first laminate sheet and the cover member and such that the connection parts would be at positions to be approximately contained within the first sheet portion of the cover member when viewed in a plan view of the biosensor, to thereby produce a biosensor.

### Examples 2 to 5, and Comparative Example 1

Biosensors were produced in the same manner as in Example 1, except that unlike in Example 1, a first tacky layer was formed using the living body tacky agents 2 to 6 instead of the living body tacky agent 1.

### Evaluation of Characteristics of Biosensor

Adhesion reliability was measured using the biosensors of Examples and Comparative Example described above. Adhesion reliability was evaluated by performing a tensile endurance test. The measurement results are indicated in Table 2.

### Adhesion Reliability

### 1. Tensile Endurance Test

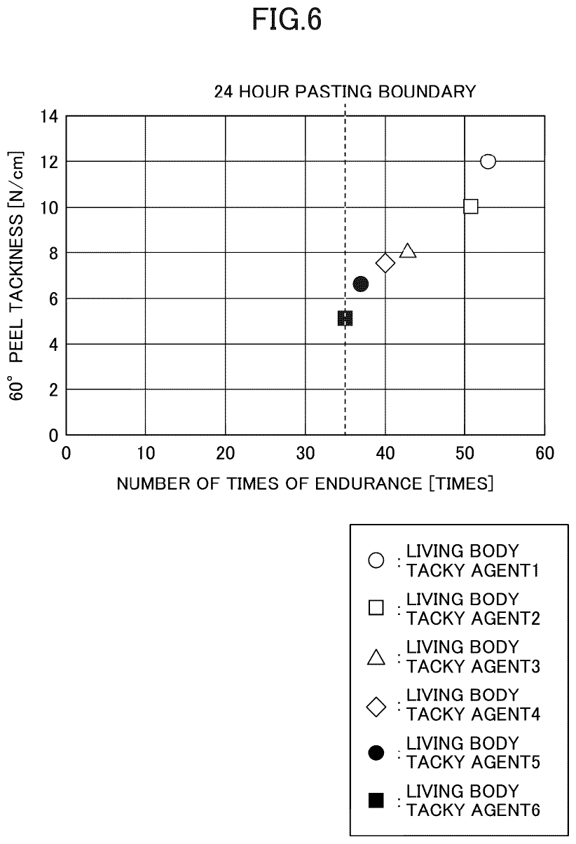

A high-performance artificial skin model (product name: Bioskin Plate, obtained from Beaulax Corporation, hereinafter referred to as bioskin plate) was used as a substitute for the skin, and the biosensor was pasted and fixed on the bioskin plate. The bioskin plate had been stored at 22° C. and 50 RH % for 3 days in order to stabilize the internal moisture content. The bioskin plate on which the biosensor was pasted was set in a small-size tabletop endurance tester (a surface-state tensile tester, obtained from Yuasa System Co., Ltd.). The bioskin plate was set so as to be strained by 20%, and was stretched repeatedly every 3 seconds until peeling occurred at an end of the biosensor, to investigate the number of times of stretching until peeling occurred. When the number of times of repeated stretching of the bioskin plate was 36 times or more, the biosensor was evaluated as being able to acquire biological signals approximately stably approximately without being peeled from the surface of the living body even if the surface of the living body would undergo deformation or the like due to a body movement during 24 hours of use of the biosensor by being pasted on the living body (24 hour pasting boundary). FIG. 6 shows the relationship between the number of times taken until the biosensor of each Example and Comparative Example was peeled and the 60° peel tackiness of the laminate of each of the living body tacky agents 1 to 6 used in Examples and Comparative Example.

TABLE 2

| | | | | | | | Comp. |
|---|---|---|---|---|---|---|---|
| | | Ex. 1 | Ex. 2 | Ex. 3 | Ex. 4 | Ex. 5 | Ex. 1 |
| Living body tacky | | L.B. | L.B. | L.B. | L. B. | L.B. | L.B. |
| agent type | | Tac. Agt. | Tac. Agt. | Tac. Agt. | Tac. Agt. | Tac. Agt. | Tac. Agt. |
| | | 1 | 2 | 3 | 4 | 5 | 6 |
| Number of | Bioskin | 53 | 51 | 43 | 40 | 37 | 35 |
| times of | plate | | | | | | |
| endurance | | | | | | | |
| [times] | | | | | | | |

According to Tables 1 and 2, the tensile endurance of each Example was 37 times or more, while the tensile endurance of Comparative Example 1 was 35 times. As shown in FIG. 6, the greater the 60° peel tackiness of the laminate used in each Example and Comparative Example, the greater the number of times taken until the biosensor of each Example and Comparative Example was peeled.

Therefore, it was confirmed that the living body tacky agent according to the above embodiments exhibited a high adhesion reliability to the skin surface by having 60° peel tackiness equal to or greater than a predetermined value. Therefore, it can be concluded that use of the living body tacky agent according to the present embodiment as the first tacky layer to be provided on a surface of the upper sheet of the biosensor on the side to be pasted with the living body enables the biosensor to be effectively used for measuring an electrocardiogram continuously even when it is pasted on the skin of the subject for a long time (for example, 24 hours).

Aspects of the embodiments of the present invention are, for example, as follows.
<1> A living body tacky agent to be pasted on a living body,
   wherein the living body tacky agent is to be pasted on a living body, and
   a peel tackiness of the living body tacky agent when the living body tacky agent, which is pasted on an adherend having an Asker C hardness of 0, is peeled from the adherend at an angle of 60° between a surface of the living body tacky agent facing the adherend and a pasting surface of the adherend is 5.0 N/cm or greater.
<2> The living body tacky agent according to <1>,
   wherein a glass transition temperature of the living body tacky agent is −57° C. or higher.
<3> The living body tacky agent according to <1> or <2>,
   wherein the peel tackiness is a value measured by pasting the living body tacky agent on the adherend while pasting polyethylene terephthalate on a surface of the living body tacky agent different from the surface facing the adherend.
<4> A biosensor to be pasted on a living body to acquire a biological signal, the biosensor including:
   a sensor body configured to acquire biological information;
   an electrode connected to the sensor body; and
   a first layer member having a lower surface on which the electrode is pasted, and including a storage space in which the sensor body is stored,
   wherein the first layer member includes the living body tacky agent of any one of <1> to <3>.
<5> The biosensor according to <4>,
   wherein the first layer member includes:
      a substrate; and
      a first tacky layer provided on a surface of the substrate facing the living body,
   wherein the first tacky layer includes the living body tacky agent.
<6> The biosensor according to <4> or <5>,
   wherein the first layer member includes:
      a cover member including a storage space in which the sensor body is stored and an opening defining the storage space; and
      an upper tacky layer via which the cover member and the living body tacky agent are pasted, and
   wherein the living body tacky agent is provided on a side of the cover member where the opening is opened, and has a through-hole at a position corresponding to the storage space.
<7> The biosensor according to any one of <4> to <6>, further including:
   a second layer member pasted over the lower surface of the first layer member so as to expose the electrode and cover the sensor body,
   wherein the second layer member has a second tacky layer on a surface thereof opposite to the first layer member.
<8> The biosensor according to <7>,
   wherein the first layer member, the electrode, and the second layer member form a pasting surface to be pasted on the living body.
<9> The biosensor according to <7>,
   wherein the second tacky layer is the living body tacky agent of any one of <1> to <3>, and
   the first layer member, the electrode, and the second layer member form a pasting surface to be pasted on the living body.

This application claims priority based on Japan Patent Application No. 2022-118682, filed with the Japanese Patent Office on Jul. 26, 2022, and

Japanese Patent Application No. 2023-42213, filed with the Japan Patent Office on Mar. 16, 2023, the entire contents of which are incorporated herein.

### REFERENCE SIGNS LIST

1 biosensor
2 skin
10 first layer member
11 cover member
12 upper sheet
12a, 121a, 122a through-hole
20, 20A, 20B electrode
20a facing part
20b exposed part
30 sensor part
31 flexible substrate
32 sensor body
33A connection part
33A, 33B connection part
34 battery
40 second layer member
41 second substrate
42 lower tacky layer
43 second tacky layer
111 protrusion
111a recess
121 first substrate
122 first tacky layer
123 upper tacky layer
321 component mounting part
322 battery attaching part

1. A living body tacky agent to be pasted on a living body,
wherein a peel tackiness of the living body tacky agent when the living body tacky agent, which is pasted on an adherend having an Asker C hardness of 0, is peeled from the adherend at an angle of 60° between a surface of the living body tacky agent facing the adherend and a pasting surface of the adherend is 5.0 N/cm or greater.

2. The living body tacky agent according to claim 1,
wherein a glass transition temperature of the living body tacky agent is −57° C. or higher.

3. The living body tacky agent according to claim 1,
wherein the peel tackiness is a value measured by pasting the living body tacky agent on the adherend while pasting polyethylene terephthalate on a surface of the living body tacky agent different from the surface facing the adherend.

4. A biosensor to be pasted on a living body to acquire a biological signal, the biosensor comprising:
a sensor body configured to acquire biological information;
an electrode connected to the sensor body; and
a first layer member having a lower surface on which the electrode is pasted, and including a storage space in which the sensor body is stored,
wherein the first layer member includes the living body tacky agent of claim 1.

5. The biosensor according to claim 4,
wherein the first layer member includes:
a substrate; and
a first tacky layer provided on a surface of the substrate facing the living body,
wherein the first tacky layer is the living body tacky agent.

6. The biosensor according to claim 5,
wherein the first layer member includes:
a cover member including a storage space in which the sensor body is stored and an opening defining the storage space; and
an upper tacky layer via which the cover member and the living body tacky agent are pasted, and
wherein the living body tacky agent is provided on a side of the cover member where the opening is opened, and has a through-hole at a position corresponding to the storage space.

7. The biosensor according to claim 4, further comprising:
a second layer member pasted over the lower surface of the first layer member so as to expose the electrode and cover the sensor body,
wherein the second layer member has a second tacky layer on a surface thereof opposite to the first layer member.

8. The biosensor according to claim 7,
wherein the first layer member, the electrode, and the second layer member form a pasting surface to be pasted on the living body.

9. The biosensor according to claim 7,
wherein the second tacky layer is the living body tacky agent of claim 1, and
the first layer member, the electrode, and the second layer member form a pasting surface to be pasted on the living body.
